(19) United States
(12) Patent Application Publication
(10) Pub. No.: US 2016/0051225 A1
(43) Pub. Date: Feb. 25, 2016

(54) ULTRASONIC TRANSDUCERS

(71) Applicant: Samsung Electronics Co., Ltd., Suwon-si (KR)

(72) Inventors: Dongkyun KIM, Suwon-si (KR); Seogwoo HONG, Yongin-si (KR)

(21) Appl. No.: 14/660,167

(22) Filed: Mar. 17, 2015

(30) Foreign Application Priority Data
Aug. 21, 2014 (KR) 10-2014-0109043

(51) Int. Cl. A61B 8/00; A61B 8/08; A61B 8/14; G01S 15/89

(52) U.S. Cl. CPC A61B 8/4494; G01S 15/8925; A61B 8/5207; A61B 8/54; A61B 8/461; A61B 8/14

(57) ABSTRACT

An ultrasonic transducer may comprise: a substrate; a barrier wall on the substrate; a diaphragm fixed to the barrier wall and defining a cavity, together with the barrier wall and the substrate; a pair of electrodes, facing each other with the cavity therebetween, configured to receive a driving voltage for driving the diaphragm; and/or a plurality of posts in the cavity and having a height smaller than that of the barrier wall.

### CROSS-REFERENCE TO RELATED APPLICATION(S)

This application claims priority from Korean Patent Application No. 10-2014-0109043, filed on Aug. 21, 2014, in the Korean Intellectual Property Office (KIPO), the entire contents of which are incorporated herein by reference.

### BACKGROUND

1. Field

Some example embodiments may relate generally to ultrasonic transducers for transmitting ultrasonic waves and/or for receiving ultrasonic waves.

2. Description of Related Art

Ultrasonic devices such as ultrasonic diagnosis devices may display tomograms of a target object, such as a person or an animal, on a monitor and may provide information necessary for diagnosis of the target object by radiating ultrasonic waves toward the target object and/or detecting echo signals reflected from the target object.

Probes of ultrasonic diagnosis devices may be equipped with ultrasonic transducers capable of converting electric signals into ultrasonic signals and vice versa. Such an ultrasonic transducer may include a plurality of ultrasonic cells arranged in one or two dimensions. Micromachined ultrasonic transducers (MUTs) may be used as ultrasonic cells. According to the converting method, micromachined ultrasonic transducers may be classified as piezoelectric micromachined ultrasonic transducers (pMUT), capacitive micromachined ultrasonic transducers (cMUT), magnetic micromachined ultrasonic transducers (mMUT), etc.

For example, a capacitive micromachined ultrasonic transducer may include a diaphragm vibrating according to a potential difference. Boundary portions of the diaphragm may be fixedly supported. A high degree of ultrasonic output power may be obtained by increasing the displacement of the diaphragm. Deformation of the diaphragm may be restricted at the fixed boundary portions of the diaphragm both in a translational direction and a rotational direction. However, this restriction of the deformation of the diaphragm at the fixed boundary portions may be an obstacle to increasing the ultrasonic output power and/or receiving sensitivity of the ultrasonic transducer.

### SUMMARY

Some example embodiments may provide ultrasonic transducers in which diaphragms are less restricted.

Some example embodiments may provide ultrasonic transducers capable of improving ultrasonic output power.

Some example embodiments may provide ultrasonic transducers capable of improving receiving sensitivity.

In some example embodiments, an ultrasonic transducer may comprise: a substrate; a barrier wall on the substrate; a diaphragm fixed to the barrier wall and defining a cavity, together with the barrier wall and the substrate; a pair of electrodes, facing each other with the cavity therebetween, configured to receive a driving voltage for driving the diaphragm; and/or a plurality of posts in the cavity and having a height smaller than that of the barrier wall.

In some example embodiments, the diaphragm may be freely supported on the posts.

In some example embodiments, a height difference between the barrier wall and the posts may be set in such a manner that a difference between atmospheric pressure and an internal pressure of the cavity causes the diaphragm to deform and make contact with upper ends of the posts.

In some example embodiments, a height difference between the barrier wall and the posts may be set in such a manner that when a direct current (DC) bias voltage is applied to the pair of electrodes, the diaphragm deforms and makes contact with upper ends of the posts.

In some example embodiments, a height difference between the barrier wall and the posts may range from several nanometers to several tens of nanometers.

In some example embodiments, a gap between the barrier wall and outer posts, which are among the posts and adjacent to the barrier wall, may be greater than a gap between the posts.

In some example embodiments, a plurality of ultrasonic cells may be in the cavity. Each of the ultrasonic cells may be defined by three of more of the posts.

In some example embodiments, the ultrasonic transducer may further comprise a plurality of ultrasonic elements comprising the ultrasonic cells. The ultrasonic elements may be separated from each other by the barrier wall.

In some example embodiments, the substrate may comprise trenches at positions corresponding to boundaries of the ultrasonic elements to electrically separate the ultrasonic elements from each other and to prevent propagation of bulk acoustic waves.

In some example embodiments, a gap between the barrier wall and outer posts, which are among the posts and adjacent to the barrier wall, may be greater than a gap between the posts.

In some example embodiments, the ultrasonic transducer may further comprise: a plurality of ultrasonic elements comprising the ultrasonic cells; and/or a plurality of ultrasonic element groups comprising the ultrasonic elements. The ultrasonic element groups may be separated from each other by the barrier wall.

In some example embodiments, the substrate may comprise trenches at positions corresponding to boundaries of the ultrasonic elements to electrically separate the ultrasonic elements from each other and to prevent propagation of bulk acoustic waves.

In some example embodiments, in each of the ultrasonic element groups, a gap between the barrier wall and the ultrasonic elements adjacent to the barrier wall may be greater than or equal to a gap between the ultrasonic elements.

In some example embodiments, the ultrasonic elements may be two-dimensionally arranged in each of the ultrasonic element groups. Boundary element columns and boundary element rows of the ultrasonic elements adjacent to the barrier wall may be filled with deactivated ultrasonic elements.

### BRIEF DESCRIPTION OF THE DRAWINGS

The above and/or other aspects and advantages will become more apparent and more readily appreciated from the following detailed description of example embodiments, taken in conjunction with the accompanying drawings, in which:

FIG. 1 is a schematic view illustrating the structure of an ultrasonic device according to some example embodiments;

FIG. 2 is a plan view illustrating an ultrasonic transducer according to some example embodiments;

FIG. 3 is a cross-sectional view illustrating the ultrasonic transducer of FIG. 2 according to some example embodiments;

FIG. 4 is a plan view illustrating an arrangement of a plurality of posts according to some example embodiments;

FIG. 5 is a plan view illustrating an arrangement of a plurality of posts according to some example embodiments;

FIG. 6 is a cross-sectional view illustrating the ultrasonic transducer of FIG. 2 when a diaphragm is in contact with posts according to some example embodiments;

FIG. 7 is a cross-sectional view illustrating the ultrasonic transducer of FIG. 2 when the diaphragm is operated by a driving voltage according to some example embodiments;

FIG. 8 is a cross-sectional view illustrating the displacement of a fixedly supported diaphragm;

FIG. 9 is a cross-sectional view illustrating the displacement of the diaphragm in the ultrasonic transducer of FIG. 3 according to some example embodiments;

FIG. 10 is a graph illustrating the displacement of a circular flat plate having a fixedly supported circumferential boundary and the displacement of a circular flat plate having a freely supported circumferential boundary when the circular flat plates are under a uniform load;

FIG. 11 is a plan view illustrating an ultrasonic transducer according to some example embodiments;

FIG. 12 is a cross-sectional view illustrating the ultrasonic transducer of FIG. 11 according to some example embodiments;

FIG. 13 is a cross-sectional view illustrating the ultrasonic transducer of FIG. 11 when a diaphragm is in contact with posts according to some example embodiments;

FIG. 14 is a plan view illustrating an ultrasonic transducer according to some example embodiments; and

FIG. 15 is a plan view illustrating an ultrasonic transducer according to some example embodiments.

### DETAILED DESCRIPTION

Example embodiments will now be described more fully with reference to the accompanying drawings. Embodiments, however, may be embodied in many different forms and should not be construed as being limited to the embodiments set forth herein. Rather, these example embodiments are provided so that this disclosure will be thorough and complete, and will fully convey the scope to those skilled in the art. In the drawings, the thicknesses of layers and regions may be exaggerated for clarity.

It will be understood that when an element is referred to as being “on,” “connected to,” “electrically connected to,” or “coupled to” to another component, it may be directly on, connected to, electrically connected to, or coupled to the other component or intervening components may be present. In contrast, when a component is referred to as being “directly on,” “directly connected to,” “directly electrically connected to,” or “directly coupled to” another component, there are no intervening components present. As used herein, the term “and/or” includes any and all combinations of one or more of the associated listed items.

It will be understood that although the terms first, second, third, etc., may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section. For example, a first element, component, region, layer, and/or section could be termed a second element, component, region, layer, and/or section without departing from the teachings of example embodiments.

Spatially relative terms, such as “beneath,” “below,” “lower,” “above,” “upper,” and the like may be used herein for ease of description to describe the relationship of one component and/or feature to another component and/or feature, or other component(s) and/or feature(s), as illustrated in the drawings. It will be understood that the spatially relative terms are intended to encompass different orientations of the device in use or operation in addition to the orientation depicted in the figures.

The terminology used herein is for the purpose of describing particular example embodiments only and is not intended to be limiting of example embodiments. As used herein, the singular forms “a,” “an,” and “the” are intended to include the plural forms as well, unless the context clearly indicates otherwise. It will be further understood that the terms “comprises,” “comprising,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and should not be interpreted in an idealized or overly formal sense unless expressly so defined herein.

Reference will now be made to example embodiments, which are illustrated in the accompanying drawings, wherein like reference numerals may refer to like components throughout.

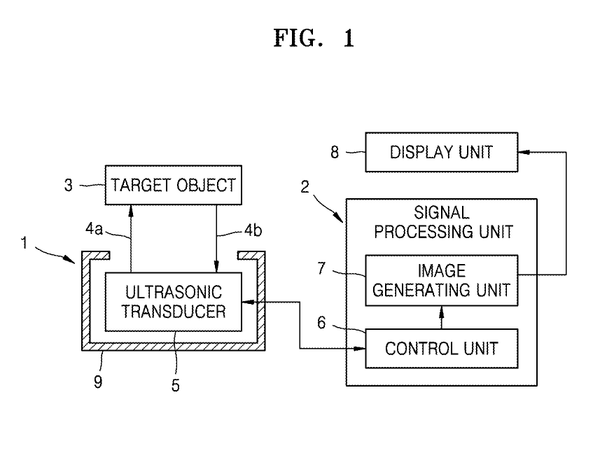

FIG. 1 is a schematic view illustrating the structure of an ultrasonic device according to some example embodiments. Referring to FIG. 1, the ultrasonic device includes an ultrasonic probe 1 and a signal processing unit 2. The ultrasonic probe 1 includes an ultrasonic transducer 5 which transmits ultrasonic waves 4a to a target object (such as a human body) 3 and receives ultrasonic waves 4b reflected from the target object 3. The ultrasonic transducer 5 is disposed in a housing 9.

The signal processing unit 2 controls the ultrasonic probe 1 and produces images of the target object 3 based on echo signals which are detected using the ultrasonic probe 1 and provide information about the target object 3. The signal processing unit 2 may include a control unit 6 and an image generating unit 7. The control unit 6 may control the ultrasonic transducer 5 so as to transmit and receive ultrasonic waves 4a and 4b. After the control unit 6 determines the position of the target object 3 to be irradiated with ultrasonic waves and the intensity of the ultrasonic waves, the control unit 6 may control the ultrasonic transducer 5. Those of ordinary skill in the art to which example embodiments belong will understand that the control unit 6 may additionally control general operations of the ultrasonic probe 1. For diagnosis, the ultrasonic transducer 5 may receive echo ultrasonic waves reflected from the target object 3 and may generate an echo ultrasonic signal based on the ultrasonic waves. The image generating unit 7 receives the echo ultrasonic signal and generates ultrasonic images of the target object 3 by using the echo ultrasonic signal. General procedures for generating ultrasonic images by using an echo ultrasonic signal are apparent to those of ordinary skill in the art to which example embodiments belong and, thus, descriptions thereof will not be provided. Ultrasonic images may be displayed on a display unit 8.

For example, the signal processing unit 2 may be configured as a processor including an array in which a plurality of logic gates are arranged, or as a combination of a general-purpose microprocessor and a memory storing a program executable on the general-purpose microprocessor. Those of ordinary skill in the art to which example embodiments belong will understand that the signal processing unit 2 may be configured by using any other proper hardware.

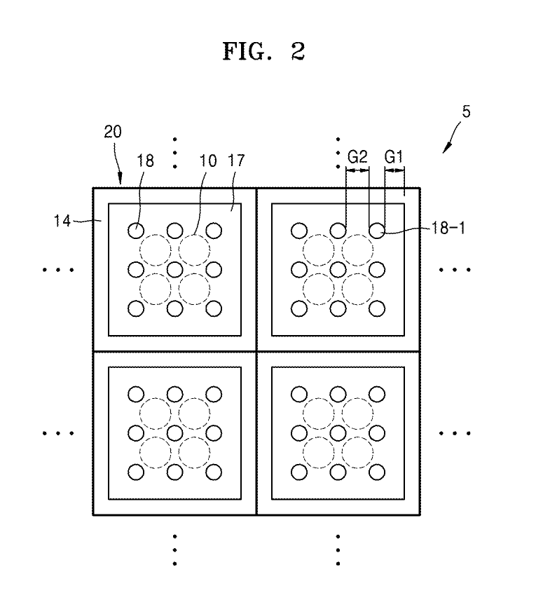

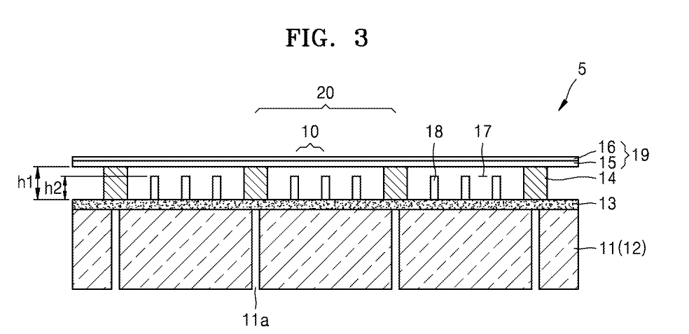

FIG. 2 is a plan view illustrating the ultrasonic transducer 5 according to some example embodiments. FIG. 3 is a cross-sectional view illustrating the ultrasonic transducer 5 of FIG. 2 according to some example embodiments.

Referring to FIGS. 2 and 3, the ultrasonic transducer 5 includes a plurality of ultrasonic cells 10. The ultrasonic cells 10 may be arranged in one or two dimensions. Each of the ultrasonic cells 10 is an ultrasonic transducer and may be a piezoelectric micromachined ultrasonic transducer (pMUT), a capacitive micromachined ultrasonic transducer (cMUT), a magnetic micromachined ultrasonic transducer (mMUT), etc. In some example embodiments, the ultrasonic cells 10 are capacitive micromachined ultrasonic transducers (cMUT). Since piezoelectric micromachined ultrasonic transducers (pMUT) use piezoelectric elements, the manufacturing of small piezoelectric micromachined ultrasonic transducers (pMUT) is limited. However, capacitive micromachined ultrasonic transducers (cMUT) are several tens of microns (μm) in size. Since it is possible to manufacture capacitive micromachined ultrasonic transducers (cMUT) through a series of semiconductor processes, a relatively large number of the ultrasonic cells 10 may be arranged in a given region when compared to the case of arranging piezoelectric micromachined ultrasonic transducers (pMUT). Therefore, a high degree of diagnostic precision may be obtained, and high-resolution images may be obtained for diagnosis.

Each capacitive micromachined ultrasonic transducer may be manufactured by forming a lower electrode 12, an insulation layer 13, and a barrier wall 14 defining a cavity 17 on a substrate 11, and disposing a diaphragm 19 on the barrier wall 14. The diaphragm 19 may include a vibration membrane 15 and an upper electrode 16. For example, the upper electrode 16 may be deposited on the vibration membrane 15. If the substrate 11 is a low-resistive substrate, the substrate 11 may function as the lower electrode 12. Examples of the low-resistive substrate include silicon substrates, and the low-resistive substrate may be doped with a conductive material.

Referring to FIG. 3, a capacitor is formed by the lower electrode 12, the diaphragm 19, and the cavity 17 disposed therebetween. If a direct current (DC) bias voltage is applied between the pair of upper and lower electrodes 16 and 12, an electrostatic force (Coulomb's force) causes the diaphragm 19 to move by a certain amount of displacement, thus the diaphragm 19 is pulled toward the lower electrode 12. The diaphragm 19 stops at a position where a reaction by internal stress of the diaphragm 19 is balanced with the electrostatic force. In this state, if an alternating current (AC) pulse voltage is applied, the diaphragm 19 starts to vibrate and produces ultrasonic waves. In a state where the ultrasonic cells 10 are moved by a certain (initial) amount of displacement under the influence of a DC bias voltage Vbias, if an external ultrasonic pressure is applied to the diaphragm 19, the displacement of the diaphragm 19 is varied. This variation of the displacement of the diaphragm 19 changes electrostatic capacity. The receiving of ultrasonic waves may be carried out in the way of detecting such a variation of electrostatic capacity. That is, if a capacitive micromachined ultrasonic transducer is used, both the transmission and receiving of ultrasonic waves are possible.

The ultrasonic transducer 5 may further include a driving substrate (not shown) disposed on a lower side of the substrate 11. A driving circuit (not shown) configured to drive the ultrasonic cells 10, and a receiving circuit (not shown) configured to receive echo ultrasonic waves from the ultrasonic cells 10 may be provided on the driving substrate. The driving substrate includes a first electrode (not shown) electrically connected to the upper electrode 16, and a second electrode (not shown) electrically connected to the lower electrode 12. In this structure, an AC pulse voltage and a DC bias voltage may be applied to the upper electrode 16 and the lower electrode 12.

In some example embodiments, the ultrasonic cells 10 are disposed in the cavity 17 defined by the barrier wall 14, the diaphragm 19, and the substrate 11. That is, the ultrasonic cells 10 are located in the cavity 17 formed as a single continuous region. The ultrasonic cells 10 are defined (distinguished) by a plurality of posts 18 arranged in the cavity 17.

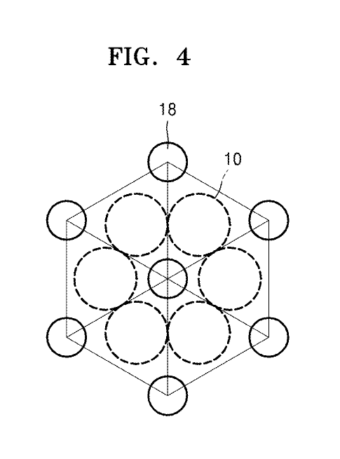

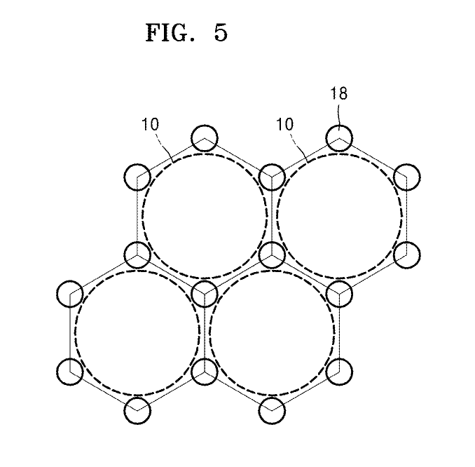

For example, referring to FIG. 2, the posts 18 are arranged in a rectangular pattern in the cavity 17. In this case, each of the ultrasonic cells 10 may be defined by four neighboring posts 18 as indicated by a dashed circle. FIG. 4 illustrates an arrangement of the posts 18 according to some example embodiments. Referring to FIG. 4, an ultrasonic cell 10 is defined by three neighboring posts 18. FIG. 5 illustrates an arrangement of the posts 18 according to some example embodiments. Referring to FIG. 5, an ultrasonic cell 10 is defined by six neighboring posts 18. However, example embodiments are not limited thereto. That is, the posts 18 may be arranged in various manners.

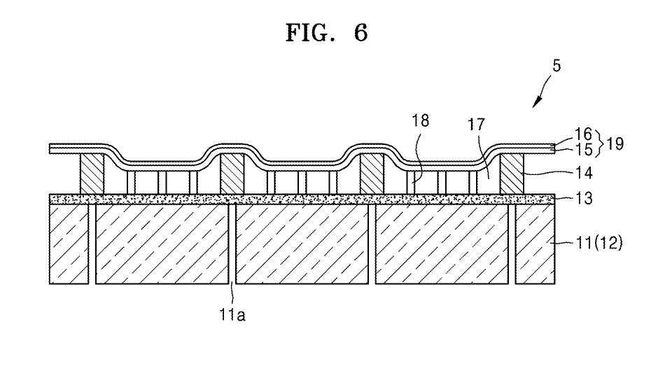

The diaphragm 19 is fixed to the barrier wall 14 forming sidewalls of the cavity 17. The height h2 of the posts 18 is smaller than the height h1 of the barrier wall 14. In a state where a DC bias voltage is not applied, the diaphragm 19 may be deformed by a difference between atmospheric pressure and the internal pressure of the cavity 17. In this case, as shown in FIG. 6, the diaphragm 19 may make contact with the posts 18 and may be supported by the posts 18 as shown in FIG. 6. The height h2 of the posts 18 and the height h1 of the barrier wall 14 may be determined to satisfy the above-mentioned condition. For example, the height difference (h1-h2) between the posts 18 and the barrier wall 14 may be several nanometers (nm) to several tens of nanometers (nm).

However, example embodiments are not limited thereto. When a DC bias voltage is applied, the diaphragm 19 may make contact with the posts 18 and may be supported on the posts 18 as shown in FIG. 6. In this case, the amount of DC bias voltage may be determined in consideration of the height h2 of the posts 18 and the height h1 of the barrier wall 14. On the other hand, after determining the amount of DC bias voltage, the height h2 of the posts 18 and the height h1 of the barrier wall 14 may be determined in such a manner that when the diaphragm 19 is deformed by the DC bias voltage, the diaphragm 19 may make contact with the posts 18 and may be supported on the posts 18.

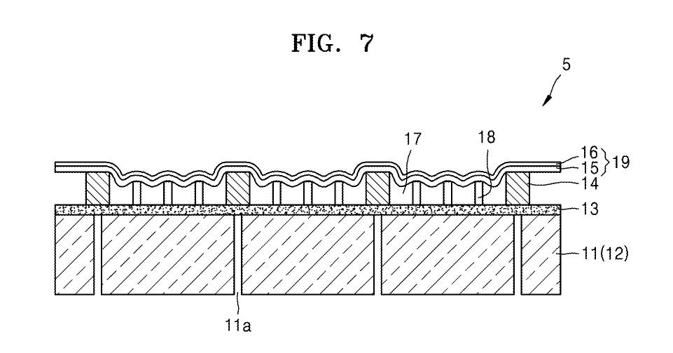

In the above-described structure of the ultrasonic transducer 5, the diaphragm 19 may be fixed to the barrier wall 14 defining the cavity 17 and may be freely supported on the posts 18. That is, the ultrasonic cells 10 share the cavity 17, and portions of the diaphragm 19 respectively corresponding to the ultrasonic cells 10 are freely supported by the posts 18. In the state shown in FIG. 6, if an AC pulse voltage is applied to the pair of lower and upper electrodes 12 and 16 or an external sound pressure is applied to the diaphragm 19, the diaphragm 19 is deformed as shown in FIG. 7.

For example, as shown in FIG. 2, the ultrasonic transducer 5 may include a plurality of two-dimensionally arranged ultrasonic elements 20. Each of the ultrasonic elements 20 may include a plurality of ultrasonic cells 10. The ultrasonic elements 20 are separated by the barrier wall 14. Each of the ultrasonic elements 20 may be operated as a single unit. In addition, two or more of the ultrasonic elements 20 may be operated as a single unit.

The sound pressure of ultrasonic waves of the ultrasonic transducer 5 is dependent on the volume variation of the cavity 17. For this reason, the diaphragm 19 is configured in such a manner that the displacement of the diaphragm 19 is large in response to a given AC pulse voltage.

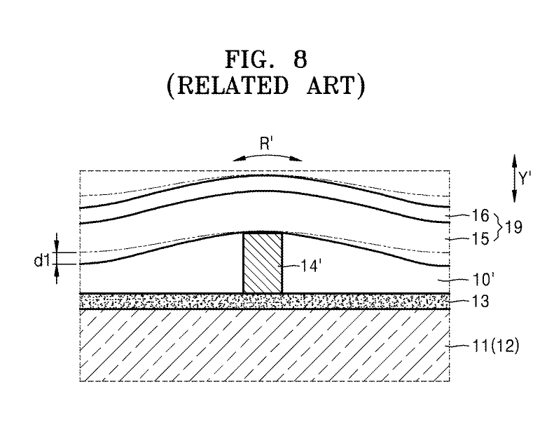

FIG. 8 is a cross-sectional view illustrating the displacement of a fixedly supported diaphragm 19. Referring to FIG. 8, in an ultrasonic transducer of the related art, each ultrasonic cell 10′ is surrounded by a barrier wall 14′. The diaphragm 19 is fixed to the barrier wall 14′ forming each ultrasonic cell 10′. In the ultrasonic transducer 5 illustrated in FIGS. 2 and 3, if the posts 18 are extended to have the same height as that of the barrier wall 14 and surround the ultrasonic cells 10, each of the posts 18 may function like the barrier wall 14′ shown in FIG. 8. In the structure of the related art, the displacement of the diaphragm 19 at the barrier wall 14′ in a translational direction Y′ is “zero,” and the slope of the diaphragm 19 at the barrier wall 14′ in a rotational direction R′ is also “zero.” That is, a displacement d1 of the diaphragm 19 causing a pressure variation of a cavity 17 is restricted by the barrier wall 14′. In addition, since the barrier wall 14′ occupies a relatively large area, the number of the ultrasonic cells 10′ per unit area is relatively small. That is, fill-factor is low.

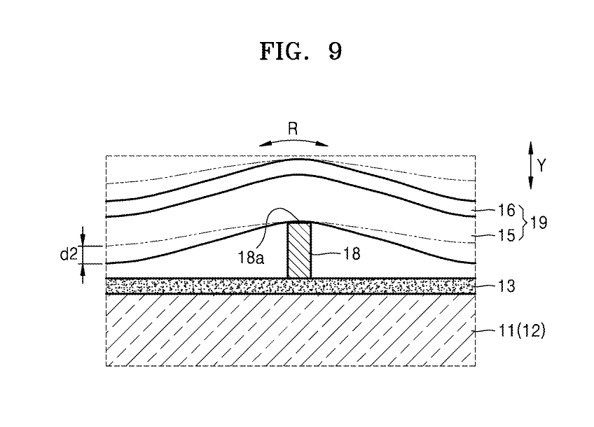

FIG. 9 is a cross-sectional view illustrating the displacement of the diaphragm 19 in the ultrasonic transducer 5 of FIG. 3 according to some example embodiments. Referring to FIG. 9, the diaphragm 19 is not fixed to an upper end 18a of a post 18 but is freely supported on the upper end 18a of the post 18. Owing to the freely supporting structure of the ultrasonic transducer 5, even though the displacement of the diaphragm 19 at the post 18 in a translational direction Y is “zero,” the displacement of the diaphragm 19 at the post 18 in a rotational direction R is not “zero.” Although the diaphragm 19 is supported on the post 18, the diaphragm 19 may be freely deformed in the rotational direction R. Thus, a displacement d2 of the diaphragm 19 is less restricted by the post 18 when compared to the case of an ultrasonic transducer having the fixedly supporting structure shown in FIG. 8. Therefore, the displacement d2 of the diaphragm 19 in the ultrasonic transducer 5 having a freely supporting structure may be larger than the displacement d1 of the diaphragm 19 in the ultrasonic transducer having a fixedly supporting structure and, thus, a larger amount of sound pressure may be obtained in the ultrasonic transducer 5 having a freely supporting structure. In addition, a relatively high degree of receiving sensitivity may be obtained when receiving ultrasonic waves. In addition, since the posts 18 are arranged inside the barrier wall 14 surrounding the ultrasonic elements 20, a higher fill-factor may be obtained when compared to the case of a related-art ultrasonic transducer having a fixedly supporting structure.

The displacement d2 of the freely supported diaphragm 19 and the displacement d1 of the fixedly supported diaphragm 19 may be indirectly compared by contrasting a displacement e2 of a circular flat plate having a freely supported circumferential boundary and a displacement e1 of a circular flat plate having a fixedly supported circumferential boundary. The displacements e1 and e2 may be expressed by the following equations:

$$e 1 ( r ) = {\frac{p {a}^{4}}{64 D} [ 1 - ( \frac{r}{a} ) ]}^{2}$$

$$e 2 ( r ) = \frac{p {a}^{4}}{64 D} [ 1 - {( \frac{r}{a} )}^{2} ] [ 3 - {( \frac{r}{a} )}^{2} ]$$

where ‘p’ denotes a load, ‘a’ denotes the diameter of a circular flat plate, ‘r’ denotes a distance measured from the center of the circular flat plate, and ‘D’ denotes the flexural rigidity of the circular flat plate and may be expressed by the following equation.

$$D = \frac{{Eh}^{3}}{12 ( 1 - {v}^{2} )}$$

where ‘E’ denotes the Young's modulus of the circular flat plate, ‘h’ denotes the thickness of the circular flat plate, and ‘v’ denotes the Poisson's ratio of the circular flat plate.

From the above-mentioned equations, the displacements e1 and e2 at the centers of the circular flat plates (that is, r=0) may be simply expressed by the following equations:

$$e 1 ( 0 ) = \frac{p {a}^{4}}{64 D}$$

$$e 2 ( 0 ) = \frac{3 p {a}^{4}}{64 D} = 3 e 1 ( 0 )$$

Therefore, even under the same load condition, the displacement e2 of the freely supported circular flat plate at the center thereof is three times the displacement e1 of the fixedly supported circular flat plate at the center thereof. The above-mentioned results of the calculation may not be exactly applied to the displacement of the diaphragm 19. However, if the height of the cavity 17 is sufficiently high in the ultrasonic transducer 5 of some example embodiments in which the diaphragm 19 is freely supported on the posts 18, the volume of the cavity 17 may be varied much more when compared to the case in which the diaphragm 19 is fixedly supported on the barrier wall 14′ forming the ultrasonic cells 10′. Therefore, the ultrasonic transducer 5 of some example embodiments may generate ultrasonic waves having a higher sound pressure and have an improved degree of receiving sensitivity.

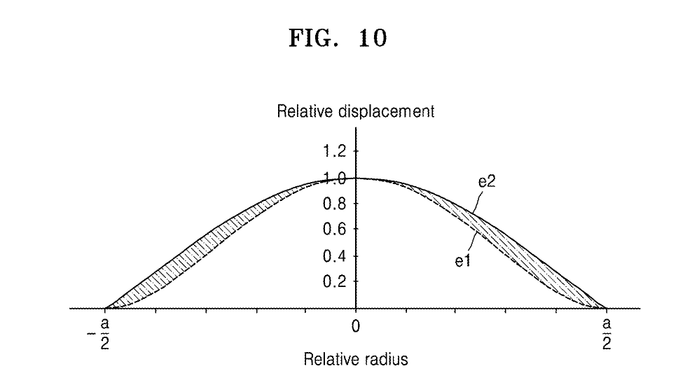

FIG. 10 is a graph illustrating the displacement e1 of a circular flat plate having a fixedly supported circumferential boundary and the displacement e2 of a circular flat plate having a freely supported circumferential boundary when the circular flat plates are under a uniform load. The graph of FIG. 10 was obtained by performing a simulation under the condition that the amounts of displacement of the circular flat plates at the centers thereof are equal (that is, e1(0)=e2(0)). Referring to FIG. 10, in the fixedly supported structure, since the slope of the circumferential boundary (a/2, −a/2) in a rotational direction R′ is “zero,” the displacement e1 gradually increases from the circumferential boundary (a/2, −a/2). However, in the freely supported structure, since the slope of the circumferential boundary (a/2, −a/2) in a rotational direction R is not limited to “zero,” the displacement e2 increases in a relatively steep manner from the circumferential boundary (a/2, −a/2). Therefore, the freely supported structure may lead to a large amount of displacement when compared to the fixedly supported structure.

If the displacement e2 shown in FIG. 10 is assumed as the displacement of the diaphragm 19 of the ultrasonic transducer 5, the volume variation of the cavity 17 in the freely supported structure is larger than the volume variation of the cavity 17 in the fixedly supported structure by a value corresponding hatched areas in FIG. 10. According to results of calculation, the volume variation of the cavity 17 in the freely supported structure is greater than the volume variation of the cavity 17 in the fixedly supported structure by about 33%.

Referring back to FIG. 2, since the diaphragm 19 is fixed to the barrier wall 14, the displacement of the diaphragm 19 is much affected by a fixedly supporting structure as it goes toward the barrier wall 14. Therefore, if the ultrasonic cells 10 are sufficiently separated from the barrier wall 14, the displacement of the diaphragm 19 may be less affected by the barrier wall 14. In some example embodiments, since the ultrasonic cells 10 are defined by the plurality of posts 18, a gap G1 between the barrier wall 14 and outer posts 18-1 adjacent to the barrier wall 14 is set to be greater than a gap G2 between the posts 18. This may reduce the effect of the barrier wall 14 on the displacement of the diaphragm 19 and, thus, the displacement of the diaphragm 19 may become uniform over the plurality of ultrasonic cells 10 disposed in the ultrasonic elements 20.

Referring to FIG. 3, trenches 11a are formed in the substrate 11 for insulating the ultrasonic elements 20 from each other and preventing the propagation of bulk acoustic waves. The trenches 11a are formed in the substrate 11 at positions corresponding to boundaries of the ultrasonic elements 20. Since the ultrasonic elements 20 are electrically insulated from each other, the ultrasonic elements 20 may be individually operated, and since the propagation of bulk acoustic waves between the ultrasonic elements 20 is prevented, crosstalk between ultrasonic detection signals of the ultrasonic elements 20 may be prevented to improve sensitivity. For example, the trenches 11a may extend in a direction from the lower surface to the upper surface of the substrate 11.

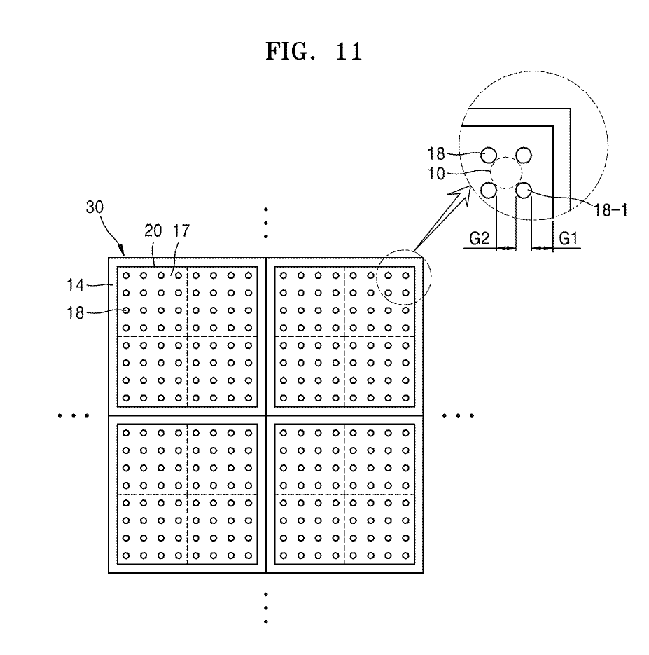

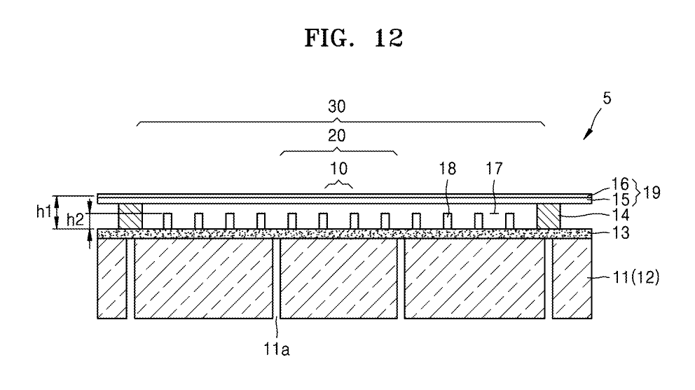

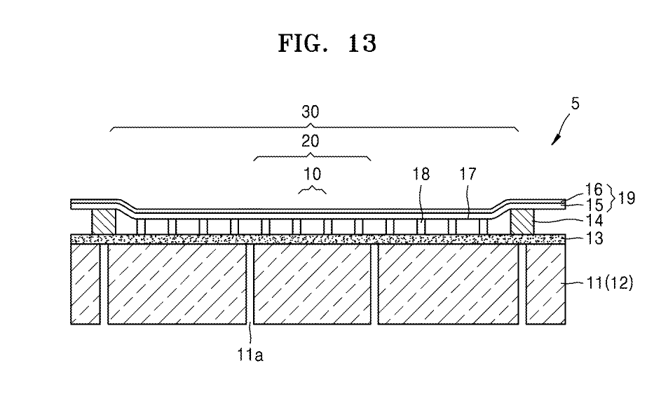

FIG. 11 is a plan view illustrating an ultrasonic transducer 5 according to some example embodiments. FIG. 12 is a cross-sectional view illustrating the ultrasonic transducer 5 of FIG. 11 according to some example embodiments. FIG. 13 is a cross-sectional view illustrating the ultrasonic transducer 5 of FIG. 11 when a diaphragm 19 is in contact with posts 18 according to some example embodiments.

Referring to FIGS. 11 to 13, a plurality of ultrasonic element groups 30 each include a plurality of ultrasonic elements 20. The ultrasonic element groups 30 are separated by a barrier wall 14. A cavity 17 is defined by the barrier wall 14, a substrate 11, and the diaphragm 19. Each of the ultrasonic elements 20 forms a single operation unit. In addition, two or more of the ultrasonic elements 20 may form a single operation unit. Trenches 11a are formed in the substrate 11 to insulate the ultrasonic elements 20 from each other and prevent the propagation of bulk acoustic waves. The barrier wall 14 does not exist between the ultrasonic elements 20. That is, a plurality of ultrasonic elements 20 included in each of the single ultrasonic element groups 30 shares the cavity 17. The ultrasonic elements 20 may be distinguished from each other by the trenches 11a. The posts 18 are arranged in the cavity 17 to define the ultrasonic cells 10. Three or more neighboring posts 18 may define a single ultrasonic cell 10.

The height h2 of the posts 18 is smaller than the height h1 of the barrier wall 14. In a state where a DC bias voltage is not applied, the diaphragm 19 may be deformed by a difference between atmospheric pressure and the internal pressure of the cavity 17. In this case, the diaphragm 19 may make contact with the posts 18 and may be supported by the posts 18 as shown in FIG. 13. The height h2 of the posts 18 and the height h1 of the barrier wall 14 may be determined to satisfy the above-mentioned condition. For example, the height difference (h1-h2) between the posts 18 and the barrier wall 14 may be several nanometers (nm) to several tens of nanometers (nm).

However, example embodiments are not limited thereto. When a DC bias voltage is applied, the diaphragm 19 may make contact with the posts 18 and may be supported on the posts 18 as shown in FIG. 13. In this case, the amount of DC bias voltage may be determined in consideration of the height h2 of the posts 18 and the height h1 of the barrier wall 14. On the other hand, after determining the amount of DC bias voltage, the height h2 of the posts 18 and the height h1 of the barrier wall 14 may be determined in such a manner that when the diaphragm 19 is deformed by the DC bias voltage, the diaphragm 19 may make contact with the posts 18 and may be supported on the posts 18.

The structure may reduce an area occupied by the barrier wall 14 and, thus, guarantee a higher fill-factor compared to the case of the ultrasonic transducer 5 shown in FIG. 2. In addition, since the displacement of the diaphragm 19 is less limited by the barrier wall 14, ultrasonic waves having a higher sound pressure may be generated, and a higher degree of receiving sensitivity may be obtained. As described above, a gap G1 between the barrier wall 14 and outer posts 18-1 adjacent to the barrier wall 14 is set to be greater than a gap G2 between the posts 18. Therefore, the barrier wall 14 on which the diaphragm 19 is fixedly supported may have less effect on the displacement of the diaphragm 19, and the displacement of the diaphragm 19 may be uniform over the ultrasonic cells 10 of the ultrasonic element groups 30.

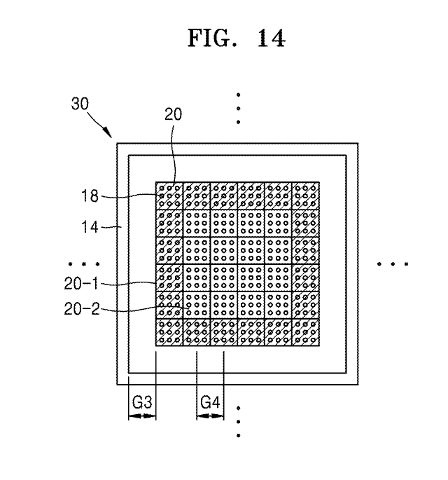

FIG. 14 is a plan view illustrating an ultrasonic transducer 5 according to some example embodiments. Referring to FIG. 14, an ultrasonic element group 30 includes a plurality of ultrasonic elements 20. Since a diaphragm 19 is fixed supported on a barrier wall 14, the displacement of the diaphragm 19 is much affected by the fixedly supporting structure between the barrier wall 14 and the diaphragm 19 as it goes close to the barrier wall 14. Therefore, operational characteristics of boundary ultrasonic elements 20-1 adjacent to the barrier wall 14 may be different from operation characteristics of inner ultrasonic elements 20-2 distant from the barrier wall 14. This difference in operational characteristics may be removed by sufficiently separating the boundary ultrasonic elements 20-1 from the barrier wall 14. For example, a gap G3 between the boundary ultrasonic elements 20-1 and the barrier wall 14 may be equal to or greater than a gap G4 between the ultrasonic elements 20. In this case, the fixedly supporting structure between the barrier wall 14 and the diaphragm 19 may have less effect on the displacement of the diaphragm 19 over the boundary ultrasonic elements 20-1 and, thus, the difference in the operational characteristics of the boundary ultrasonic elements 20-1 and the inner ultrasonic elements 20-2 may be decreased.

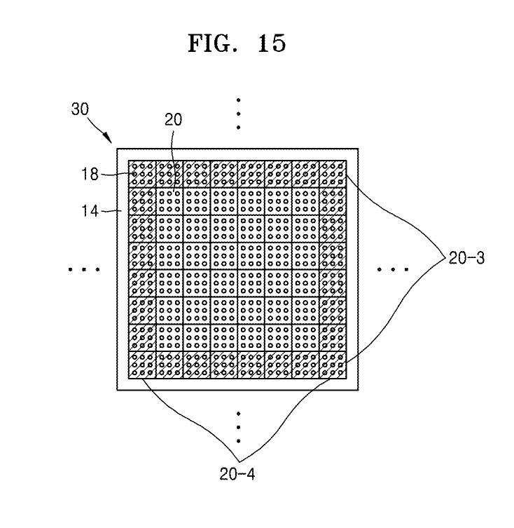

In another method for decreasing the difference in the operational characteristics of the ultrasonic elements 20 according to the distance from the barrier wall 14, ultrasonic elements (refer to reference numerals 20-3 and 20-4 in FIG. 15) adjacent to the barrier wall 14 are deactivated. FIG. 15 is a plan view illustrating an ultrasonic transducer 5 according to some example embodiments. Referring to FIG. 15, an ultrasonic element group 30 includes a plurality of ultrasonic elements 20 arranged in two-dimensional form. A diaphragm 19 is fixed to a barrier wall 14. Among the ultrasonic elements 20, boundary element columns 20-3 and boundary element rows 20-4 are filled with deactivated elements. The term “deactivated elements” refers to dummy elements that are not operated. For example, the deactivated elements may be formed when the ultrasonic transducer 5 is manufactured by omitting first and second electrodes (not shown) for applying driving voltages to upper and lower electrodes 16 and 12. In addition, the deactivated elements may be provided by not operating the boundary element columns 20-3 and the boundary element rows 20-4. In other words, when generating ultrasonic waves, a driving voltage may not be applied to the boundary element columns 20-3 and the boundary element rows 20-4, or when receiving ultrasonic waves, receiving signals of the boundary element columns 20-3 and the boundary element rows 20-4 may not be used.

It should be understood that the example embodiments described herein should be considered in a descriptive sense only and not for purposes of limitation. Descriptions of features or aspects within each embodiment should typically be considered as available for other similar features or aspects in other embodiments.

While some example embodiments have been described with reference to the figures, it will be understood by those of ordinary skill in the art that various changes in form and details may be made therein without departing from the spirit and scope as defined by the following claims.

What is claimed is:

1. An ultrasonic transducer, comprising:
a substrate;
a barrier wall on the substrate;
a diaphragm fixed to the barrier wall and defining a cavity, together with the barrier wall and the substrate;
a pair of electrodes, facing each other with the cavity therebetween, configured to receive a driving voltage for driving the diaphragm; and
a plurality of posts in the cavity and having a height smaller than that of the barrier wall.

2. The ultrasonic transducer of claim 1, wherein the diaphragm is freely supported on the posts.

3. The ultrasonic transducer of claim 2, wherein a height difference between the barrier wall and the posts is set in such a manner that a difference between atmospheric pressure and an internal pressure of the cavity causes the diaphragm to deform and make contact with upper ends of the posts.

4. The ultrasonic transducer of claim 2, wherein a height difference between the barrier wall and the posts is set in such a manner that when a direct current (DC) bias voltage is applied to the pair of electrodes, the diaphragm deforms and makes contact with upper ends of the posts.

5. The ultrasonic transducer of claim 2, wherein a height difference between the barrier wall and the posts ranges from several nanometers to several tens of nanometers.

6. The ultrasonic transducer of claim 1, wherein a gap between the barrier wall and outer posts, which are among the posts and adjacent to the barrier wall, is greater than a gap between the posts.

7. The ultrasonic transducer of claim 1, wherein a plurality of ultrasonic cells are in the cavity, and
wherein each of the ultrasonic cells is defined by three of more of the posts.

8. The ultrasonic transducer of claim 7, wherein the ultrasonic transducer further comprises a plurality of ultrasonic elements comprising the ultrasonic cells, and
wherein the ultrasonic elements are separated from each other by the barrier wall.

9. The ultrasonic transducer of claim 8, wherein the substrate comprises trenches at positions corresponding to boundaries of the ultrasonic elements to electrically separate the ultrasonic elements from each other and to prevent propagation of bulk acoustic waves.

10. The ultrasonic transducer of claim 8, wherein a gap between the barrier wall and outer posts, which are among the posts and adjacent to the barrier wall, is greater than a gap between the posts.

11. The ultrasonic transducer of claim 7, wherein the ultrasonic transducer further comprises:
a plurality of ultrasonic elements comprising the ultrasonic cells; and
a plurality of ultrasonic element groups comprising the ultrasonic elements;
wherein the ultrasonic element groups are separated from each other by the barrier wall.

12. The ultrasonic transducer of claim 11, wherein the substrate comprises trenches at positions corresponding to boundaries of the ultrasonic elements to electrically separate the ultrasonic elements from each other and to prevent propagation of bulk acoustic waves.

13. The ultrasonic transducer of claim 11, wherein in each of the ultrasonic element groups, a gap between the barrier wall and the ultrasonic elements adjacent to the barrier wall is greater than or equal to a gap between the ultrasonic elements.

14. The ultrasonic transducer of claim 11, wherein the ultrasonic elements are two-dimensionally arranged in each of the ultrasonic element groups, and
wherein boundary element columns and boundary element rows of the ultrasonic elements adjacent to the barrier wall are filled with deactivated ultrasonic elements.
